(19) United States
(12) Patent Application Publication
(10) Pub. No.: US 2011/0294680 A1
(43) Pub. Date: Dec. 1, 2011

(54) METHODS AND COMPOSITIONS FOR PREDICTING SUCCESS IN ADDICTIVE SUBSTANCE CESSATION AND FOR PREDICTING A RISK OF ADDICTION

(75) Inventors: Jed E. Rose, Durham, NC (US); George R. Uhl, Towson, MD (US)

(73) Assignee: Duke University, Durham, NC (US)

(21) Appl. No.: 12/918,940

(22) PCT Filed: Feb. 20, 2009
(86) PCT No.: PCT/US09/34694

§ 371 Date: Oct. 13, 2010

Related U.S. Application Data
(60) Provisional application No. 61/030,728, filed on Feb. 22, 2008.

(51) Int. Cl. C40B 30/04; C12Q 1/68; C07H 21/04

(52) U.S. Cl. 506/9; 536/23.1; 435/6.11

(57) ABSTRACT

The present invention relates to genetic polymorphisms that are associated with dependence on an addictive substance. In particular, the present invention relates to a method for predicting success in addictive substance cessation in a subject, such as predicting success in nicotine cessation. In some embodiments, nicotine cessation is accompanied by a nicotine replacement source and/or an antidepressant. The invention further provides a method for identifying a subject who has an increased risk of becoming dependent on an addictive substance. In some embodiments, the addictive substance is nicotine. Also provided are isolated nucleic acid molecules containing the polymorphisms and reagents for detecting the polymorphic nucleic acid molecules.

### STATEMENT REGARDING FEDERALLY SPONSORED RESEARCH OR DEVELOPMENT

This invention was made with United States government support under P50CA/DA84718 awarded by the National Institutes of Health Intramural Research Program, NIDA, DHSS. The United States government has certain rights in the invention.

### FIELD OF THE INVENTION

The present invention relates to methods for predicting an ability of a subject to quit using an addictive substance, as well as to methods for predicting a subject's risk of becoming dependent on an addictive substance.

### BACKGROUND OF THE INVENTION

Substance dependence, both legal and controlled, represents one of the most important preventable causes of illness and death in modern society. The path to addiction generally begins with a voluntary use of one or more addictive substances such as tobacco, alcohol, narcotics or any of a variety of other addictive substances. With extended use of the addictive substance, a voluntary ability to abstain from the addictive substance is compromised in many subjects. As such, substance addiction is generally characterized by compulsive substance craving, habitual substance seeking and substance use that persists even in the face of negative consequences. Substance addiction is also characterized in many cases by withdrawal symptoms.

Nicotine, as found in tobacco, is one such addictive substance. Worldwide, tobacco use causes nearly 5 million deaths per year, with current trends showing that tobacco use will cause more than 10 million deaths annually by 2020 (World Health Organization (2002) The World Health Report 2002: Reducing Risks, Promoting Healthy Life). In the United States, cigarette smoking is a leading preventable cause of death and is responsible for about one in five deaths annually, or about 438,000 deaths per year (Centers for Disease Control and Prevention (2005) Morbid. Mortal. Wkly Rep. 54:625-628). Nearly 21% of U.S. adults (45.1 million people) are current cigarette smokers (Centers for Disease Control and Prevention (2005) Morbid. Mortal. Wkly Rep. 54:1121-1124). Among adult smokers, 70% report that they want to quit completely (id.), and more than 40% try to quit each year (Substance Abuse and Mental Health Services Administration (2006) Results from the 2005 National Survey on Drug Use and Health: National Findings (Office of Applied Studies, NSDUH Series H-30, DHHS Publication No. SMA 06-4194)). Quitting smoking even after prolonged use of tobacco has substantial health benefits. Unfortunately, a majority of subjects who report quit attempts report that they failed to abstain permanently.

A primary goal of therapy or treatment of substance addiction is to reduce the amount and/or rate of intake of the addictive substance over time, as well as to reduce the rate of relapse. Individuals afflicted with an addictive condition who succeed in obtaining a reduction or complete cessation of intake of the addictive substance remain at a substantial risk to relapse during the course of their lifetimes. To completely eradicate the addictive condition over the subject's lifetime often requires life-long administration of therapy, be it pharmacological, behavioral or both.

Substance cessation programs typically address both pharmacological and psychological factors. Vulnerability to substance dependence, however, is a substantially heritable complex disorder (Karkowski et al. (2000) Am. J. Med. Genet. 96:665-670; Tsuang et al. (1998) Arch. Gen. Psychiatry 55:967-972; True et al. (1999) Am. J. Med. Genet. 88:391-397). Classical genetic studies also indicate that individual differences in an ability to successfully quit using the addictive substance are substantially heritable, but differ from those that influence aspects of dependence (Xian et al. (2003) Nicotine Tob. Res. 5:245-254). Therefore, there remains a need for methods to predict a likelihood of successful cessation of an addictive substance, as well as for methods to predict a potential for substance dependence or addiction.

### SUMMARY OF THE INVENTION

The present invention relates to an identification of novel sets of single nucleotide polymorphisms (SNPs), unique combinations of such SNPs and haplotypes of SNPs that are associated with 1) an increased ability to quit using an addictive substance or 2) an increased risk of becoming dependent on an addictive substance. The SNPs disclosed herein are useful as targets for designing diagnostic reagents based on genetic profiling for use in determining a subject's genetic predisposition to 1) quit using an addictive substance or 2) become dependent on an addictive substance.

In a first aspect of the invention, a method for predicting success in addictive substance cessation in a subject includes detecting a SNP at one or more polymorphic sites of genes (or gene sequences) described herein in a nucleic acid complement of the subject, where the presence of the SNP is correlated with an increased rate of success in addictive substance cessation.

In one embodiment of this aspect, a method for predicting success in nicotine cessation in a subject using a nicotine replacement source and/or an antidepressant includes detecting a SNP at one or more polymorphic sites of genes (or gene sequences) described herein in a nucleic acid complement of the subject, where the presence of the SNP is correlated with an increased rate of success in nicotine cessation in the subject using a nicotine replacement source and/or an antidepressant. The nicotine replacement source can be a nicotine patch, a nicotine gum, a nicotine inhaler and/or a nicotine nasal spray, and the antidepressant can be bupropion.

In a second aspect of the invention, a method of determining a subject's genetic predisposition to becoming dependent on an addictive substance includes obtaining a nucleic acid sample from the subject and determining an identity of one or more bases (nucleotides) at polymorphic sites of genes (or gene sequences) described herein, where the presence of a particular base is correlated with an increased risk of becoming dependent on the addictive substance.

In a third aspect of the invention, a method for developing an individualized treatment regimen for addictive substance cessation in a subject dependent on an addictive substance includes detecting a SNP at one or more polymorphic sites of genes (or gene sequences) described herein in a nucleic acid complement of the subject, where the presence of the SNP is correlated with an individualized treatment regimen through a genetic association between specific SNPs, the particular addictive substance the subject is dependent on and rates of success in addictive substance cessation in individuals utilizing behavioral modification and/or pharmacological therapy. The addictive substance can be nicotine.

A fourth aspect of the invention includes allele-specific oligonucleotides that hybridize to reference or variant alleles of genes including a SNP or to the complement thereof. These oligonucleotides can be probes or primers.

### DETAILED DESCRIPTION OF THE INVENTION

The present invention provides SNPs associated with quit success of an addictive substance or an increased risk of becoming dependent on an addictive substance, nucleic acid molecules containing the SNPs disclosed herein, methods and reagents for detecting the SNPs disclosed herein, uses of the SNPs disclosed herein for developing detection reagents, and assays or kits utilizing such reagents. The addictive substance-associated SNPs disclosed herein therefore are useful for diagnosing, screening and evaluating quit success or predisposition to becoming dependent on the addictive substance.

The genomes of all organisms undergo spontaneous mutation throughout evolution, generating variant forms of progenitor genetic sequences (Gusella (1986) Ann. Rev. Biochem. 55:831-854). A variant form may confer an evolutionary advantage or disadvantage relative to a progenitor form or may be neutral. In some instances, the variant form of the progenitor genetic sequence confers an evolutionary advantage to organisms and is eventually incorporated into the DNA of many or most organisms and effectively becomes the progenitor form.

In addition, the effects of the variant form may be both beneficial and detrimental, depending on the circumstances. For example, a heterozygous sickle cell mutation confers resistance to malaria, but a homozygous sickle cell mutation is usually lethal. In many cases, both progenitor and variant forms of a genetic sequence survive and co-exist in a species population. The coexistence of multiple forms of a genetic sequence gives rise to genetic polymorphisms, including SNPs.

Approximately 90% of all polymorphisms in the human genome are SNPs. SNPs are single base positions in DNA at which different alleles, or alternative nucleotides, exist in a population. SNP position (interchangeably referred to herein as SNP, SNP site, SNP locus, SNP marker or marker) is usually preceded and followed by highly conserved sequences of the allele (e.g., sequences that vary in less than 1/100 or 1/1000 members of the population). A subject may be homozygous or heterozygous for the allele at each SNP position. A SNP can, in some instances, be referred to as a “cSNP,” which denotes that the nucleotide sequence containing the SNP is an amino acid coding sequence.

A SNP also may arise from a substitution of one nucleotide for another at the polymorphic site. Substitutions can be transitions or transversions. A transition is the replacement of one purine by another purine, or one pyrimidine by another pyrimidine. A transversion is the replacement of a purine by a pyrimidine or a pyrimidine by a purine. A SNP may also be a single base insertion or deletion variant referred to as an “indel” (Weber et al. (2002) Am. J. Hum. Genet. 71:854-862).

A synonymous codon change, or silent mutation SNP (terms such as “SNP,” “polymorphism,” “mutation,” “mutant,” “variation” and “variant” are used herein interchangeably), is one that does not result in a change of amino acid due to the degeneracy of the genetic code. A substitution that changes a codon coding for one amino acid to a codon coding for a different amino acid (i.e., a non-synonymous codon change) is referred to as a missense mutation. A nonsense mutation results in a type of non-synonymous codon change in which a stop codon is formed, thereby leading to premature termination of a polypeptide chain and a truncated protein. A read-through mutation is another type of non-synonymous codon change that causes the destruction of a stop codon, thereby resulting in an extended polypeptide product. While SNPs can be bi-, tri-, or tetra-allelic, the vast majority of the SNPs are bi-allelic, and are thus often referred to as “bi-allelic markers” or “di-allelic markers.”

As used herein, references to SNPs and SNP genotypes include individual SNPs and/or haplotypes, which are groups of SNPs that are generally inherited together. Haplotypes can have stronger correlations with increased risk of becoming dependent on an addictive substance compared with individual SNPs, and therefore can provide increased diagnostic accuracy in some cases (Stephens et al. (2001) Science 293:489-493).

An association study of a SNP and an increased risk of becoming dependent on an addictive substance involves determining a presence or frequency of the SNP allele(s) in biological samples from test subjects with a dependency of interest, such as nicotine dependency, and comparing the information to that of control subjects (i.e., subjects who are not dependent on the addictive substance) who are usually of similar age and race. A SNP may be screened in any biological sample obtained from a test subject and compared to like samples from control subjects, and selected for its increased occurrence in a specific or general dependency on one or more addictive substances, such as nicotine dependency. Once a statistically significant association is established between one or more SNP(s) and a dependency on an addictive substance of interest, then the region around the SNP can optionally be thoroughly screened to identify the causative genetic locus/sequence(s) (e.g., causative SNP mutation, gene, regulatory region, and the like) that influences the dependency.

Thus, the present invention pertains to a method for predicting success in addictive substance cessation in a subject, including detecting a SNP in one or more (e.g., 2, 3, 4, 5, 10, 15, 20, 25, 30, 40, 50, 60, 70, 80, 90, 100, 150, 200, 250, 300, 350, 400, 450, 500, 550, 600, 650, 700, 750, 800, 850, 900, 950, 1000 or any number in-between) nucleotide sequences set forth in SEQ ID NOs:1-14724 in a nucleic acid complement of the subject (see, Table 1). In a non-limiting example, the nucleotide sequences can be at least twenty or more of the nucleotide sequences set forth in SEQ ID NOs:1-14724. The presence of the SNP is correlated with an increased rate of success in addictive substance cessation.

By addictive substance “cessation” is intended a bringing or coming to an end; a ceasing or stopping (i.e., of use of the addictive substance). By an “increased rate” of success in addictive substance cessation is meant a higher than normal rate of ceasing or stopping use of an addictive substance by a subject, compared to the general population. In a non-limiting example, the addictive substance is nicotine. In a further embodiment, the subject presently is dependent on an addictive substance (e.g., nicotine).

By “addictive substance” is intended any substance that causes or is characterized by addiction, that is, strong physiological and/or psychological dependence on the substance. Addictive substances include, but are not limited to, nicotine; alcohol; cannabis (e.g., marijuana); stimulants, such as cocaine and amphetamines (e.g., methamphetamine and Ecstasy); hallucinogens (e.g., LSD, PCP and ketamine); depressants (e.g., diazepam and barbiturates); sleep aids (e.g., eszopiclone, ramelteon and zolpidem); psychotropic medications, such as anti-psychotics (e.g., haloperidol, loxapine, aripiprazole, and olanzapine); antidepressants (e.g., fluoxetine, nortriptyline, sertraline and bupropion); anti-anxiety agents (e.g., diazepam, alprazolam and sertraline); and narcotics, such as heroin, codeine, morphine and oxycodone. For review, see, Substance Abuse: A Comprehensive Textbook (Lowinson et al. eds. 2^{nd} ed. Lippincott Williams & Wilkins, NY, 2005).

The term “nucleic acid complement” of a subject refers to a total nucleic acid content of the subject (e.g., as found in a biological sample, such as a cell, of a subject), and includes a full set of genes (i.e., DNA), their translation products (i.e., RNA) and non-coding genetic material.

SNP genotyping to identify a subject with an increased risk of becoming dependent on an addictive substance, predicting success in addictive substance cessation in a subject, predicting success in nicotine cessation in a subject using a nicotine replacement source and/or an antidepressant, and other uses described herein, typically relies on initially establishing a genetic association between one or more (e.g., 2, 3, 4, 5, 10, 15, 20, 25, 30, 40, 50, 60, 70, 80, 90, 100, 150, 200, 250, 300, 350, 400, 450, 500, 550, 600, 650, 700, 750, 800, 850, 900, 950, 1000 or any number in-between) specific SNPs and the particular traits, habits or actions of interest.

Different study designs may be used for genetic association studies (see, e.g., Modern Epidemiology pp. 609-622 (Lippincott Williams & Wilkins 1998). One such study design is an observational study. Observational studies are most frequently carried out in which a response of subjects is not interfered with. One type of observational study is a case-control or retrospective study. In typical case-control studies, samples are collected from subjects with the habit or action of interest (cases), such as dependency on one or more addictive substances, and from individuals in whom dependency is absent (controls) in a population (target population) that conclusions are to be drawn from. Then, the possible causes of the traits, habits or actions, e.g., dependency on an addictive substance, such as nicotine, are investigated retrospectively. There may be potential confounding factors that should be taken into consideration. Confounding factors are those that are associated with both the real cause(s) of the dependency and the dependency itself, and they may include demographic information such as age, gender and ethnicity, as well as environmental factors. When confounding factors are not matched in cases and controls in a study, and are not controlled properly, spurious association results can arise. If potential confounding factors are identified, they can be controlled for by analysis methods well known to those of ordinary skill in the art.

Another study design is a genetic association study. In a genetic association study, a cause of interest to be tested is a certain allele or a SNP, or a combination of alleles or a haplotype from several SNPs. Thus, tissue specimens (e.g., blood) from a subject can be collected and genomic DNA genotyped for the SNP(s) of interest. In addition to the trait or habit of interest, other information such as demographic (e.g., age, gender and ethnicity), clinical and environmental information that may influence the outcome of the trait or habit can be collected to further characterize and define the sample set. In many cases, this information is known to be associated with dependency and/or SNP allele frequencies. There are likely gene-environment and/or gene-gene interactions as well.

After all the relevant trait, habit and/or action information and genotypic information is obtained, statistical analyses can be carried out to determine if there is any significant correlation between the presence of an allele or a genotype with the substance dependency of the subject. Data inspection and cleaning can be first performed before carrying out statistical tests for genetic association. Epidemiological and clinical data of the samples can be summarized by descriptive statistics with tables and graphs. Data validation can be performed to check for data completion, inconsistent entries, and outliers. Chi-squared tests and t-tests (Wilcoxon rank-sum tests if distributions are not normal) then can be used to check for significant differences between cases and controls for discrete and continuous variables, respectively. To ensure genotyping quality, Hardy-Weinberg disequilibrium tests can be performed on cases and controls separately. Significant deviation from Hardy-Weinberg equilibrium in both cases and controls for individual markers can be indicative of genotyping errors.

To test whether an allele of a SNP is associated with the case or control status of a trait or habit, one of ordinary skill in the art can compare allele frequencies in cases and controls. Standard chi-squared tests and Fisher exact tests can be carried out on a 2×2 table (2 SNP alleles by 2 outcomes in the categorical trait of interest). To test whether genotypes of a SNP are associated, chi-squared tests can be carried out on a 3×2 table (3 genotypes by 2 outcomes). Score tests can also carried out for genotypic association to contrast the three genotypic frequencies (major homozygotes, heterozygotes and minor homozygotes) in cases and controls, and to look for trends using three different modes of inheritance, namely dominant (with contrast coefficients 2, −1, −1), additive (with contrast coefficients 1, 0, −1) and recessive (with contrast coefficients 1, 1, 2). Odds ratios for minor versus major alleles, and odds ratios for heterozygote and homozygote variants versus the wild-type genotypes are calculated with the desired confidence limits, usually 95%. For samples genotyped in DNA pools, t-tests assess the relationship between relative allelic frequencies in cases versus controls. To control for confounders and to test for interaction and effect modifiers, stratified analyses can be performed using stratified factors that are likely to be confounding, including demographic information such as age, ethnicity and gender, or an interacting element or effect modifier such as known major genes (e.g., nicotine metabolizing enzymes for nicotine dependency) or environmental factors such as polysubstance abuse.

In addition to performing association tests one marker at a time, haplotype association analysis can also be performed to study a number of markers that are closely linked together. Haplotype association tests may have better power than genotypic or allelic association tests when the tested markers are not the mutations causing the predisposition to dependency themselves, but are in linkage disequilibrium with such mutations. In order to perform haplotype association effectively, marker-marker linkage disequilibrium measures, both D and R², are typically calculated for the markers within a gene to elucidate the haplotype structure. Studies in linkage disequilibrium suggest that SNPs within a given gene are organized in block pattern, and a high degree of linkage disequilibrium exists within blocks and very little linkage disequilibrium exists between blocks (Daly et al. (2001) Nat. Gen. 29:232-235). Haplotype association with predisposition to dependency on an addictive substance can be performed using such blocks once they have been elucidated. Haplotype association tests can be carried out in a similar fashion as the allelic and genotypic association tests. Each haplotype in a gene is analogous to an allele in a multi-allelic marker. One of ordinary skill in the art either can compare the haplotype frequencies in cases and controls or can test genetic association with different pairs of haplotypes.

An important decision in performing genetic association tests is determining a significance level at which significant association can be declared when a p-value of the tests reaches that level. In an exploratory analysis where positive hits will be followed up in subsequent confirmatory testing, an unadjusted p-value<0.1 can be used for generating hypotheses for significant association of a SNP with certain traits or habits associated with substance dependency. Generally, a p-value<0.05 is required for a SNP for an association with a predisposition to dependency on an addictive substance, and a p-value<0.01 is required for an association to be declared. When hits are followed up in confirmatory analyses in more samples of the same source or in different samples from different sources, adjustment for multiple testing can be performed so as to avoid excess number of hits while maintaining the experiment-wise error rates at 0.05. While there are different methods known to one of ordinary skill in the art to adjust for multiple testing to control for different kinds of error rates, a commonly used method is Bonferroni correction to control the experiment-wise or family-wise error rate (Westfall et al. (1999) Multiple Comparisons and Multiple Tests (SAS Institute)). Permutation tests to control for false discovery rates also can be used (Benjamini & Hochberg (1995) J. Royal Stat. Soc. B 57:1289-1300). Monte Carlo simulation studies are especially useful in correcting for false positive results, since these tests can take into account many of the features of the true datasets without reliance on underlying statistical models. Since both genotyping and addiction status classification can involve errors, sensitivity analyses may be performed to see how odds ratios and p-values would change upon various estimates on genotyping and addiction status classification error rates.

Determining which specific nucleotide (i.e., allele) is present at each of one or more SNP positions, such as a SNP position in a nucleic acid molecule disclosed in Table 1, is referred to as SNP genotyping. The present invention therefore provides methods for SNP genotyping, such as predicting success in addictive substance cessation in a subject, predicting success in nicotine cessation in a subject using a nicotine replacement source and/or an antidepressant, identifying a subject with an increased risk of becoming dependent on an addictive substance, or other uses as described herein.

Nucleic acid samples can be genotyped to determine which alleles are present at any given genetic region (e.g., SNP position) of interest by methods well known in the art. Neighboring sequences can be used to design SNP detection reagents such as oligonucleotide probes, which may optionally be implemented in a kit format. Exemplary SNP genotyping methods are known in the art (Chen et al. (2003) Pharmacogenomics J. 3:77-96; Kwok et al. (2003) Curr. Issues Mol. Biol. 5:43-60; Shi, Am. J. Pharmacogenomics (2002) 2:197-205; and Kwok (2001) Annu. Rev. Genomics Hum. Genet. 2:235-258). Exemplary techniques for high-throughput SNP genotyping are described by Marnellos (Marnellos (2003) Curr. Opin. Drug Discov. Devel. 6:317-321). Common SNP genotyping methods include, but are not limited to, TaqMan® Gene Expression Assays (Applied Biosystems, Inc.; Foster City, Calif.), molecular beacon assays, nucleic acid arrays, allele-specific primer extension, allele-specific polymerase chain reaction (PCR), arrayed primer extension, homogeneous primer extension assays, primer extension with detection by mass spectrometry, pyrosequencing, multiplex primer extension sorted on genetic arrays, ligation with rolling circle amplification, homogeneous ligation, multiplex ligation reaction sorted on genetic arrays, restriction-fragment length polymorphism (RFLP) and single base extension-tag assays. Such methods can be used in combination with detection mechanisms such as, e g., luminescence or chemiluminescence detection, fluorescence detection, time-resolved fluorescence detection, fluorescence resonance energy transfer, fluorescence polarization, mass spectrometry and electrical detection.

Various methods for detecting polymorphisms include, but are not limited to, methods in which protection from cleavage agents is used to detect mismatched bases in RNA/RNA or RNA/DNA duplexes (Myers et al. (1985) Science 230:1242-1246; Cotton et al. (1988) Proc. Natl. Acad. Sci. USA 85:4397-4401; Saleeba et al. (1992) Meth. Enzymol. 217:286-295), comparison of the electrophoretic mobility of variant and wild-type nucleic acid molecules (Orita et al. (1989) Proc. Natl. Acad. Sci. USA 86:2766-2770; Cotton et al. (1992) Mutat. Res. 285:125-144; Hayashi et al. (1992) Genet. Anal. Tech. Appl. 9:73-79) and assaying the movement of polymorphic or wild-type fragments in polyacrylamide gels containing a gradient of denaturant using denaturing gradient gel electrophoresis (Myers et al. (1985) Nature 313:495-498). Sequence variations at specific locations also can be assessed by nuclease protection assays such as RNase and S1 protection assays or chemical cleavage methods.

In a specific embodiment, SNP genotyping is performed using the TaqMan® Assay, which also is known as a 5 nuclease assay (see, e.g., U.S. Pat. Nos. 5,210,015 and 5,538,848). The TaqMan® Assay detects accumulation of a specific amplified product during PCR. It utilizes an oligonucleotide probe labeled with a fluorescent reporter and quencher dye. When the reporter dye is excited by irradiation at an appropriate wavelength, it transfers energy to the quencher dye in the same probe via a process called fluorescence resonance energy transfer (FRET). As such, when attached to the probe, the excited reporter dye does not emit a signal. The proximity of the quencher dye to the reporter dye in the intact probe maintains a reduced fluorescence for the reporter dye. The reporter and quencher dyes can be at the 5-most and the 3-most ends of the probe, respectively, or vice versa. Alternatively, the reporter dye can be at the 5- or 3-most end of the probe, while the quencher dye is attached to an internal nucleotide, or vice versa. Alternatively, both the reporter and quencher dyes can be attached to internal nucleotides of the probe at a distance from each other, such that fluorescence of the reporter dye is reduced.

During PCR, the 5 nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target SNP-containing template, which is amplified during PCR, and the probe is designed to hybridize to the target SNP site only if a particular SNP allele is present. Preferred TaqMan primer and probe sequences can readily be determined using the SNP and associated nucleic acid sequence information provided herein. A number of computer programs, such as Primer Express (Applied Biosystems, Foster City, Calif.), can be used to rapidly obtain optimal primer/probe sets. It will be apparent to one of skill in the art that such primers and probes for detecting the SNPs of the present invention are useful in diagnostic assays for identifying a subject who has an increased risk of becoming dependent on an addictive substance, predicting success in addictive substance cessation in a subject and predicting success in nicotine cessation in a subject using a nicotine replacement source and/or an antidepressant, and can be readily incorporated into a kit format. The present invention also includes modifications of the TaqMan® Assay well known in the art, such as the use of molecular beacon probes (see, e.g., U.S. Pat. Nos. 5,118,801 and 5,312,728) and other variant formats (see, e.g., U.S. Pat. Nos. 5,866,336 and 6,117,635).

Another method for SNP genotyping is based on mass spectrometry, and takes advantage of the unique mass of each of the four nucleotides of DNA. Single nucleotide polymorphisms can be unambiguously genotyped by mass spectrometry by measuring the differences in the mass of nucleic acids having alternative SNP alleles. Matrix Assisted Laser Desorption Ionization-Time of Flight (MALDI-TOF) mass spectrometry technology can be used for extremely precise determinations of molecular mass such as SNPs (Wise et al. (2003) Rapid Commun. Mass Spectrom. 17:1195-1202). Numerous approaches to SNP analysis have been developed based on mass spectrometry. Some mass spectrometry-based methods of SNP genotyping include primer extension assays, which can also be utilized in combination with other approaches, such as traditional gel-based formats and microarrays.

SNPs also can be scored by direct DNA or RNA sequencing. A variety of automated sequencing procedures can be utilized, including sequencing by mass spectrometry (see, e.g., Int'l Patent Application Publication No. WO 94/16101; Cohen et al. (1996) Adv. Chromatogr. 36:127-162; Griffin et al. (1993) Appl. Biochem. Biotechnol. 38:147-159). The nucleic acid sequences of the present invention enable one of ordinary skill in the art to design sequencing primers for such automated sequencing procedures. Commercial instrumentation, such as the analyzers supplied by Applied Biosystems, is commonly used in the art for automated sequencing.

Sequence-specific ribozymes (see, e.g., U.S. Pat. No. 5,498,531) also can be used to score SNPs based on the development or loss of a ribozyme cleavage site. Perfectly matched sequences can be distinguished from mismatched sequences by nuclease cleavage digestion assays or by differences in melting temperature. If the SNP affects a restriction enzyme cleavage site, the SNP can be identified by alterations in restriction enzyme digestion patterns, and the corresponding changes in nucleic acid fragment lengths determined by gel electrophoresis. In some assays, the size of the amplification product is detected and compared to the length of a control sample. For example, deletions and insertions can be detected by a change in size of the amplified product compared to a control genotype.

In further embodiments, the present invention provides methods for predicting success in nicotine cessation in a subject using a nicotine replacement source and/or an antidepressant. The methods include detecting a SNP in one or more (e.g., 2, 3, 4, 5, 10, 15, 20, 25, 30, 40, 50, 60, 70, 80, 90, 100, 150, 200, 250, 300, 350, 400, 450, 500, 550, 600, 650, 700, 750, 800, 850, 900, 950, 1000 or any number in-between) nucleotide sequences set forth in SEQ ID NOs:1-14724 as disclosed in the nucleotide sequences set forth in SEQ ID NOs:1-14724 in the nucleic acid complement of the subject (see, Table 1). In a non-limiting example, the nucleotide sequences can be at least twenty or more of the nucleotide sequences set forth in SEQ ID NOs:1-14724. The presence of the SNP is correlated with an increased rate of success in nicotine cessation in a subject using the nicotine replacement source and/or the antidepressant.

By “nicotine replacement source” is intended a source of nicotine separate or apart from tobacco (e.g., an isolated and/or purified source of nicotine). An exemplary nicotine replacement source is a nicotine patch (e.g., Habitrol™, Nicoderm CQ™ and Nicotrol™) which releases a constant amount of nicotine into the body. Unlike nicotine in tobacco smoke, which passes rapidly into the blood through the lining of the lungs, nicotine in a nicotine patch takes about an hour to pass through the layers of skin and into the subject's blood. An additional nicotine replacement source is nicotine gum (e.g., Nicorette™ gum), which delivers nicotine to the brain more quickly than a patch. However, unlike the nicotine in tobacco smoke, the nicotine in the gum takes several minutes to reach the brain, making the “hit” less intense with the gum than with a cigarette. Yet another nicotine replacement source is a nicotine lozenge (e.g., Commit™ lozenge), which comes in the form of a hard candy and releases nicotine as it slowly dissolves in the mouth of a subject.

A nicotine nasal spray (e.g., Nicotrol™ nasal spray) is another example of a nicotine replacement source. Nicotine nasal spray, dispensed from a pump bottle similar to over-the-counter decongestant sprays, relieves cravings for a cigarette, as the nicotine is rapidly absorbed through the nasal membranes and reaches the bloodstream faster than any other nicotine replacement therapy (NRT) product. Yet another example of a nicotine replacement source is a nicotine inhaler (e.g., Nicotrol™ inhaler), which generally consists of a plastic cylinder containing a cartridge that delivers nicotine when a subject puffs on it. Although similar in appearance to a cigarette, a nicotine inhaler delivers nicotine into the mouth, not the lungs, and the nicotine enters the body much more slowly than the nicotine in tobacco smoke. The term “antidepressant” includes bupropion hydrochloride (e.g., Zyban™ or Wellbutrin™)

In addition, the present invention pertains to a method for identifying a subject with an increased risk of becoming dependent on an addictive substance, including detecting a SNP in one or more (e.g., 2, 3, 4, 5, 10, 15, 20, 25, 30, 40, 50, 60, 70, 80, 90, 100, 150, 200, 250, 300, 350, 400, 450, 500, 550, 600, 650, 700, 750, 800, 850, 900, 950, 1000 or any number in-between) nucleotide sequences set forth in SEQ ID NOs:1-14724 in a nucleic acid complement of the subject (see, Table 1). In a non-limiting example, the nucleotide sequences can be at least twenty or more of the nucleotide sequences set forth in SEQ ID NOs:1-14724. The presence of the SNP is correlated with an increased risk of becoming dependent on the addictive substance.

By an “increased risk” of becoming dependent on an addictive substance is intended a subject that is identified as having a higher than normal chance of developing a dependency to an addictive substance, compared to the general population. The term “becoming dependent” (i.e., on or to an addictive substance) refers to exhibiting dependence or dependency, a state in which there is a compulsive or chronic need for the addictive substance. Thus, a subject dependent on an addictive substance exhibits compulsive use of the substance despite significant problems resulting from such use. Hallmarks of dependency include, but are not limited to, taking a substance longer or in larger amounts than planned, repeatedly expressing a desire or attempting unsuccessfully to cut down or regulate use of a substance, continuing use in the face of acknowledged substance-induced physical or mental problems, tolerance and withdrawal.

Furthermore, the present invention provides methods for developing an individualized treatment regimen for addictive substance cessation in a subject dependent on an addictive substance, including detecting a SNP in one or more (e.g., 2, 3, 4, 5, 10, 15, 20, 25, 30, 40, 50, 60, 70, 80, 90, 100, 150, 200, 250, 300, 350, 400, 450, 500, 550, 600, 650, 700, 750, 800, 850, 900, 950, 1000 or any number in-between) nucleotide sequences set forth in SEQ ID NOs:1-14724 in a nucleic acid complement of the subject (see, Table 1). In a non-limiting example, the nucleotide sequences can be at least twenty or more of the nucleotide sequences set forth in SEQ ID NOs:1-14724. The presence of one or more SNPs is correlated with an individualized treatment regimen by establishing a genetic association between specific SNPs, the particular addictive substance the subject is dependent on and rates of success in addictive substance cessation in individuals utilizing behavioral modification and/or pharmacological therapy (e.g., replacement therapy). In a non-limiting example, the addictive substance is nicotine. In a further embodiment, the subject presently is dependent on an addictive substance (e.g., nicotine).

In a further embodiment, the present invention provides isolated nucleic acid molecules that contain one or more SNPs (e.g., 2, 3, 4, 5, 10, 15, 20, 25, 30, 40, 50, 60, 70, 80, 90, 100, 150, 200, 250, 300, 350, 400, 450, 500, 550, 600, 650, 700, 750, 800, 850, 900, 950, 1000 or any number in-between), as disclosed in the nucleotide sequences set forth in SEQ ID NOs:1 to 14724. Isolated nucleic acid molecules containing one or more SNPs disclosed herein may be interchangeably referred to as “SNP-containing nucleic acid molecules.” The isolated nucleic acid molecules of the present invention also include probes and primers, which can be used for assaying the disclosed SNPs. As used herein, an “isolated nucleic acid molecule” is one that contains a SNP of the present invention, or one that hybridizes to such molecule such as a nucleic acid with a complementary sequence, and is separated from most other nucleic acids present in the natural source of the nucleic acid molecule. Moreover, an “isolated” nucleic acid molecule, such as a cDNA molecule containing a SNP of the present invention, may be substantially free of other cellular material, or culture medium when produced by recombinant techniques, or chemical precursors or other chemicals when chemically synthesized. A nucleic acid molecule can be fused to other coding or regulatory sequences and still be considered “isolated.”

Isolated nucleic acid molecules may be in the form of RNA, such as mRNA, and include in vivo or in vitro RNA transcripts of the isolated SNP-containing DNA molecules of the present invention. Isolated nucleic acid molecules according to the present invention further include such molecules produced by molecular cloning or chemical synthetic techniques or by a combination thereof (see, e.g., Sambrook & Russell, Molecular Cloning: A Laboratory Manual (Cold Spring Harbor Press, NY 2000).

Generally, an isolated SNP-containing nucleic acid molecule includes one or more SNP positions disclosed by the present invention with flanking nucleotide sequences on either side of the SNP positions. A flanking sequence can include nucleotide residues that are naturally associated with the SNP site and/or heterologous nucleotide sequences. Generally the flanking sequence is up to about 100, 80, 60, 50, 40, 30, 25, 20, 15, 10, 8, 6 or 4 nucleotides (or any other length in-between) on either side of a SNP position.

An isolated nucleic acid molecule of the present invention further encompasses a SNP-containing polynucleotide that is the product of any one of a variety of nucleic acid amplification methods, which are used to increase the copy numbers of a polynucleotide of interest in a nucleic acid sample. Such amplification methods are well known in the art and include, but are not limited to, PCR (U.S. Pat. Nos. 4,683,195 and 4,683,202), ligase chain reaction (Wu & Wallace (1989) Genomics 4:560-569; Landegren et al. (1988) Science 241:1077-1080), strand displacement amplification (U.S. Pat. Nos. 5,270,184 and 5,422,252), transcription-mediated amplification (U.S. Pat. No. 5,399,491), linked linear amplification (U.S. Pat. No. 6,027,923), and isothermal amplification methods such as nucleic acid sequence based amplification and self-sustained sequence replication (Guatelli et al. (1990) Proc. Natl. Acad. Sci. USA 87:1874-1878). Based on such methodologies, one of ordinary skill in the art readily can design primers in any suitable regions 5 and 3 to a SNP disclosed herein. Such primers can be used to amplify DNA of any length so long as it contains the SNP of interest in its sequence.

Furthermore, isolated nucleic acid molecules, particularly SNP detection reagents such as probes and primers, also can be partially or completely in the form of one or more types of nucleic acid analogs, such as peptide nucleic acid (PNA; U.S. Pat. Nos. 5,539,082; 5,527,675; 5,623,049; and 5,714,331). Nucleic acids, especially DNA, can be double-stranded or single-stranded. Single-stranded nucleic acid can be the coding strand (sense strand) or the complementary non-coding strand (anti-sense strand). DNA, RNA, or PNA segments can be assembled, e.g., from fragments of the human genome (in the case of DNA or RNA) or single nucleotides, short oligonucleotide linkers, or from a series of oligonucleotides, to provide a synthetic nucleic acid molecule. Nucleic acid molecules can be readily synthesized using the sequences provided herein as a reference. Furthermore, large-scale automated oligonucleotide/PNA synthesis (including synthesis on an array, or bead surface or other solid support) can be readily accomplished using commercially available nucleic acid synthesizers, such as the Applied Biosystems (Foster City, Calif.) 3900 High-Throughput DNA Synthesizer, and the sequence information provided herein.

The nucleic acid molecules of the present invention have a variety of uses, such as predicting success in addictive substance cessation in a subject and predicting success in nicotine cessation in a subject using a nicotine replacement source and/or an antidepressant or identifying a subject who has an increased risk of becoming dependent on an addictive substance. Additionally, the nucleic acid molecules are useful as hybridization probes, such as for genotyping SNPs in messenger RNA, cDNA, genomic DNA, amplified DNA or other nucleic acid molecules, and for isolating full-length cDNA and genomic clones as well as their orthologs.

A probe can hybridize to any nucleotide sequence along the entire length of a nucleic acid molecule provided herein. Generally, a probe of the present invention hybridizes to a region of a target sequence that encompasses a SNP position indicated in Table 1. In some instances, the probe hybridizes to a SNP-containing target sequence in a sequence-specific manner, such that it distinguishes a target sequence from other nucleotide sequences that vary from the target sequence only by the nucleotide present at the SNP site. Such a probe is particularly useful for detecting a SNP-containing nucleic acid in a test sample, or for determining which nucleotide (allele) is present at a particular SNP site (i.e., genotyping the SNP site).

A nucleic acid hybridization probe can be used for determining the presence, level, form and/or distribution of nucleic acid expression. The nucleic acid whose level is determined can be DNA or RNA. Accordingly, probes specific for the SNPs described herein can be used to assess the presence, expression and/or gene copy number in a given cell, tissue or organism. In vitro techniques for detection of mRNA include, e.g., Northern blot hybridizations and in situ hybridizations. In vitro techniques for detecting DNA include Southern blot hybridizations and in situ hybridizations. Probes can be used as part of a diagnostic test kit for identifying cells or tissues in which a SNP is present, such as by determining if a polynucleotide contains a SNP of interest.

One of ordinary skill in the art will recognize that, based on the SNP and associated sequence information disclosed herein, detection reagents can be developed and used to assay any SNP of the present invention individually or in combination, and such detection reagents can be readily incorporated into one of the established kit or system formats which are well known in the art. The terms “kits” and “systems,” as used herein in the context of SNP detection reagents, are intended to refer to such things as combinations of multiple SNP detection reagents, or one or more SNP detection reagents in combination with one or more other types of elements or components (e.g., other types of biochemical reagents, containers, packages, such as packaging intended for commercial sale, substrates to which SNP detection reagents are attached, electronic hardware components, and the like). Accordingly, the present invention further provides SNP detection kits and systems, including but not limited to, packaged probe and primer sets (e.g., TaqMan® Probe Primer Sets), arrays/microarrays of nucleic acid molecules, and beads that contain one or more probes, primers, or other detection reagents for detecting one or more SNPs of the present invention. The kits/systems optionally can include various electronic hardware components. For example, arrays (e.g., DNA chips) and microfluidic systems (e.g., lab-on-a-chip systems) provided by various manufacturers typically include hardware components. Other kits/systems (e.g., probe/primer sets) may not include electronic hardware components, but can include, e.g., one or more SNP detection reagents along with other biochemical reagents packaged in one or more containers.

A SNP detection kit typically also can contain one or more detection reagents and other components (e.g., a buffer, enzymes, such as DNA polymerases or ligases, chain extension nucleotides, such as deoxynucleotide triphosphates, positive control sequences, negative control sequences, and the like) necessary to carry out an assay or reaction, such as amplification and/or detection of a SNP-containing nucleic acid molecule. A kit can further contain means for determining the amount of a target nucleic acid, and means for comparing the amount with a standard, and can include instructions for using the kit to detect the SNP-containing nucleic acid molecule of interest. In one embodiment of the present invention, kits are provided that contain the necessary reagents to carry out one or more assays to detect one or more SNPs disclosed herein. In a non-limiting example, SNP detection kits/systems are in the form of nucleic acid arrays or compartmentalized kits, including microfluidic/lab-on-a-chip systems.

SNP detection kits/systems may contain, e.g., one or more probes, or pairs of probes, that hybridize to a nucleic acid molecule at or near each target SNP position. Multiple pairs of allele-specific probes can be included in the kit/system to simultaneously assay large numbers of SNPs, at least one of which is a SNP of the present invention. In some kits/systems, the allele-specific probes are immobilized to a substrate, such as an array or bead. For example, the same substrate can comprise allele-specific probes for detecting at least 1, at least 10, at least 100, at least 1000, at least 10,000, or at least 100,000 SNPs. The terms “arrays,” “microarrays” and “DNA chips” are used herein interchangeably to refer to an array of distinct polynucleotides affixed to a substrate such as glass, plastic, paper, nylon, or other type of membrane, filter, chip or any other suitable solid support. The polynucleotides can be synthesized directly on a surface of the substrate, or synthesized separate from the substrate and then affixed to the substrate's surface.

The following experimental examples are offered by way of illustration and not by way of limitation.

### EXAMPLES

### Example 1

### Molecular Genetics of Nicotine Dependence and Abstinence

A 520,000 SNP genome wide association in pools of DNA prepared from nicotine dependent European-American smoking cessation trial participants and control individuals was performed. Genotypes from the entire group of nicotine dependent research participants were compared to genotypes from European-American research volunteers free from any substantial lifetime use of any addictive substance. See, Uhl et al. (2007) BMC Genet. 8:10, incorporated herein by reference as if set forth in its entirety.

Experimental Subjects

Study participants of self-reported European ancestry recruited in the Raleigh-Durham metropolitan area by advertising and word of mouth provided informed consents for studies of smoking cessation, averaged age 44 and were 45% female. These participants reported an average of 25 years of smoking, displayed initial Fagerstrom Test for Nicotine Dependence (FTND; Heatherton et al. (1991) Br. J. Addict. 86:1119-1127) scores that averaged 6.4 and provided screening carbon monoxide levels that averaged 34.7.

Participants received oral mecamylamine (10 mg/day) and either active (21 mg/24 hours) or placebo nicotine skin patches for two weeks before the target quit-smoking date. After the quit-date, participants were randomly assigned to groups that received mecamylamine (10 mg/day) versus matching placebo and 21 mg/24 hours versus 42 mg/24 hours nicotine skin patch doses to test how mecamylamine might improve effectiveness of nicotine replacement therapy. Behavioral support and self-help quitting manuals were also provided. Fifty-five study participants reported continuous abstinence from smoking when assessed six weeks after the quit date. Seventy-nine participants were not abstinent at the six week time point. Data from these individuals was compared to data from 320 control study participants of self-reported European-American ancestry recruited in Baltimore by advertising and word of mouth who also provided informed consents, averaged age 31, were 36% female and reported no substantial lifetime histories of use of any addictive substance (Uhl et al. (2001) Am. J. Hum. Genet. 69:1290-1300; Smith et al. (1992) Arch. Gen. Psychiatry 49:723-727; and Persico et al. (1996) Biol. Psychiatry 40:776-784).

DNA Preparation, Pooling and Analysis

Genomic DNA was prepared from blood (Uhl et al. (2001), supra; Smith et al., supra; and Persico et al., supra), carefully quantified and combined into pools representing 13-20 individuals of the same ethnicity and phenotype. Hybridization probes were prepared from the genomic DNA pools according to the manufacture's instructions (Affymetrix Genechip Mapping Assay Manual; Affymetrix; Santa Clara, Calif.) with precautions to avoid contamination that included use of dedicated preparation rooms and hoods. Fifty nanograms of each pooled genomic DNA was digested by StyI or by NspI, ligated to appropriate adaptors and amplified using a GeneAmp PCR System 9700 (Applied Biosystems) with a 3 minute 94° C. hot start, 30 cycles of 30 seconds at 94° C., 45 seconds at 60° C., 15 seconds at 68° C., and a final 7 minute 68° C. extension. PCR products were purified (MinElute™ 96 UF kits, Qiagen; Valencia, Calif.). Polymerase chain reaction products were quantified, and 40 μg was digested for 35 minutes at 37° C. with 0.04 unit/μl DNase I. The 30-100 bp fragments resulting from DNase treatments were end-labeled using terminal deoxynucleotidyl transferase and biotinylated dideoxynucleotides and hybridized to the appropriate StyI or NspI early access Mendel® Microarrays (Affymetrix). Arrays were stained, washed and scanned according to the manufacture's instructions (Affymetrix Genechip Mapping Assay Manual) using immunopure strepavidin (Pierce, Milwaukee, Wis.), biotinylated antistreptavidin antibody (Vector Labs, Burlingame, Calif.) and R-phycoerythrin strepavidin (Molecular Probes, Eugene, Oreg.). Fluorescence intensities were quantitated using an Affymetrix array scanner as described by Uhl et al. (Uhl et al. (2001), supra).

Identification of Positive SNPs

Allele frequencies for each SNP in each DNA pool were assessed based on hybridization to the 12 “perfect match” cells on each of four arrays from replicate experiments, as described by Liu et al. (Lui et al. (2006) Am. J. Med. Genet. B Neuropsychiatr. Genet. 141:918-925) and Johnson et al. (Johnson et al. (2006) Am. J. Med. Genet. B Neuropsychiatr. Genet. 141:844-853). Briefly, each cell's value was analyzed by subtracting background fluorescence intensities and normalizing background-subtracted values to the values for the highest intensities on each array. The data from the 12 perfect match cells for A and B alleles for each SNP were averaged. To facilitate comparison of data from multiple arrays, the arctangent of the ratio between hybridization intensities for A and B alleles for each array was derived. These arctan AB values for the four replicate arrays that assessed genotype frequencies for each pool were then averaged. The mean arctan A/B ratios for nicotine dependent versus control individuals (and for quitters versus nonquitters) were then calculated. The mean arctan A/B ratio for abusers (or quitters) was then divided by the mean arctan A/B ratio for controls (or nonquitters) to form abuser/control (or quitter/nonquitter) ratios. A “t” statistic for the differences between abusers and controls or quitters and nonquitters was then generated (Liu et al. (2005) Proc. Natl. Acad. Sci. USA 102:11864-11869; Liu et al. (2006), supra; Johnson et al. (2006), supra). “Nominally significant” SNPs displayed t values with p<0.005 for nicotine dependent versus control comparisons and p<0.01 for quitter versus nonquitter comparisons, respectively. A relatively strict preplanned criterion for the first comparison that confirms genes with good confidence was thus set. A more modest criterion, with lower levels of confidence, was set for the second comparison that nominates genes that merit replication studies. Data from SNPs on sex chromosomes and SNPs whose chromosomal positions could not be adequately determined using Mapviewer (NCBI, build 35.1) or NETAFFYX (Affymetrix) were deleted.

Nicotine Dependence Variants

In preplanned assessments of the allelic variants likely to influence vulnerability to dependence on nicotine and other addictive substances, autosomal SNPs that provided convergent data with four additional abuser versus comparisons datasets were focused on. That is, SNPs that a) displayed t values with p <0.005 nominal significance in comparisons between European-American controls versus nicotine dependent research participants; b) identified genes that also displayed reproducibly-positive associations with addiction vulnerabilities in data from four other samples: i) National Institute on Drug Abuse (NIDA) African-American and European-American polysubstance abuser versus control comparisons based on 639,401 SNP comparisons with the requirement that both samples provide nominally significant results (p<0.0025 for the joint probability) and clustering so that at least three such SNPs lay within 100 Kb of each other (Liu et al. (2006), supra); ii) Japanese Genetic Investigations of Drug Abuse (JGIDA) Japanese methamphetamine abuser versus control comparisons, based on a requirement for nominal significance (p<0.05) of SNPs lying within the same genes; and iii) Collaborative Study on the Genetics of Alcoholism (COGA) alcohol dependent versus control comparisons, based on a requirement for nominal significance (p<0.05) of SNPs lying within the same genes (Johnson et al. (2006), supra); and c) produced an enhanced (e.g., lower) Monte Carlo p value for the overall association in comparisons of the smoker/control data with the four other sample sets versus the Monte Carlo p values for the data from the four other sample sets alone.

Each of the Monte Carlo simulation trials began with sampling from a database that contained the results from the smoker/control study and results from a larger database that contained data from the prior association studies in the four additional samples noted herein to which the smoker/control results were compared. For each of these 100,000 simulation trials, a randomly-selected set of SNPs was chosen and the same procedure that had been followed for the actual data was run. The number of trials for which the results from the randomly-selected set of SNPs matched or exceeded the results actually observed from the SNPs identified in the smoker/control study was tabulated. Empirical p values were calculated by dividing the number of trials for which the observed results were matched or exceeded by the total number of Monte Carlo simulation trials performed. Since this method examines the properties of the SNPs in the smoker/control dataset, assuming independence of their allele frequencies, it is relatively robust despite the uneven distribution of Affymetrix SNP markers across the genome.

Quit Success Variants

In comparing results related to successful abstinence, less stringent criteria were used. The focus was on autosomal SNPs that displayed three features: 1) they displayed t values with p<0.01 nominal significance in the dataset of successful versus unsuccessful quitters; 2) they lay within clusters of at least three such nominally positive SNPs so that each positive SNP lies within 0.1 Mb of the nearest positive SNP; and 3) they lay within genes whose functions can be inferred. These observed results were also compared to those expected by chance, based on independence of SNP allelic frequency estimates under the null hypothesis, using 10,000-100,000 Monte Carlo simulation trials on the database from the study's results, as noted herein (Uhl et al. (2001), supra).

Statistical Power

To assess the power of the present approach, the observed standard deviations and mean abuser/control differences for the SNPs that provided the largest differences between control and abuser population means were used, with the program PS (v2.1.31; Dupont & Plummer (1990) Control Clin. Trials 11:116-128) and α=0.05.

Control Comparisons

To provide a control for the possibility that the user/control and abstainer/nonabstainer differences observed at some of the clustered, reproducibly-positive SNPs were due to occult ethnic/racial differences in the frequencies of alleles at these same SNPs between abusers and controls or between abstainers and nonabstainers, the present results were compared with those that have previously been obtained from comparisons of allele frequency data in self-reported African-American versus European-American control individuals, focusing on SNPs that display ethnicity difference scores that lie in the outlying +/−2.5% of all differences.

To provide a control for the possibility that the abuser-control differences observed at many of the clustered, reproducibly-positive SNPs were due to noisy assays for these SNPs, the overlap between the clustered positive SNPs and the 2.5% of SNPs which display the largest variation between pools in data from this and other studies using the same arrays were examined

Results

Single nucleotide polymorphism allele frequency assessments displayed modest variability. Standard errors for the variation among the four replicate studies of each DNA pool were +/−0.035. Standard error for the variation among the pools studied for each phenotype group was +/−0.028. Previous validating studies for these arrays have also revealed good fits between individual and pooled genotyping, with 0.95 correlations between pooled and individually-determined genotype frequencies. The observed pool-to-pool standard deviations from these datasets thus indicate 0.94 and 1.0 power to detect 5 and 10% allele frequency differences with α=0.05 in nicotine dependent versus control comparisons and 0.45 and 0.95 power to detect 5 and 10% allele frequency differences in successful versus unsuccessful quitters.

When allele frequencies in 134 nicotine dependent versus 320 control individuals were compared, 88,937 of the 520,000 tested SNPs displayed t values that provided nominally-significant abuser versus control allele frequency differences at p<0.005. These nominally-positive SNPs are positioned near clustered-positive SNPs from four other abuser-control comparisons to extents that are greater than expected by chance. 4701 of these nominally-significant SNPs lay within 100 Kb of a cluster of nominally-positive SNPs from replicate African-American and European-American NIDA polysubstance abuser versus control comparisons. Monte Carlo p values for this convergence were 0.0002. Thus, only 2 of 10,000 Monte Carlo simulation trials that each began by selecting 88,937 random SNPs displayed so many nominally-significant results near the clustered positive results from the two NIDA samples. 2133 of the nominally-significant SNPs from the nicotine dependent versus control comparison met several criteria. They lay near clusters of positive SNPs from both NIDA samples; they lay within annotated genes; they lay within genes that are also supported by nominally-positive results from JGIDA methamphetamine abuser versus control comparisons; and they lay within genes that are also supported by nominally-positive results from COGA alcohol dependent versus control comparisons. The Monte Carlo p value for the observed degree of convergence between the nicotine and prior data was 0.018.

The results of the nicotine dependent versus control comparisons showed that allelic variants in a number of genes contribute to individual differences in vulnerability to nicotine dependence (Table 1). Genes identified include genes related to cell adhesion processes (e.g., CNTN6, LRRN1, SEMA3C, CSMD1, PTPRD, LRRN6C, and CDH13), genes related to enzymatic activity (e.g., SIPA1L2, PDE1C, PDE4D, and PRKG1), genes encoding G-protein coupled receptors (e.g., the GRM7 metabotropic glutamate receptor, the orphan GPR154 receptor and the HRH4 histamine receptor), genes involved in protein processing, transcriptional regulation genes, transporter-associated genes, ion channel genes, and structural genes.

LENGTHY TABLES
The patent application contains a lengthy table section. A copy of the table is available in electronic form from the USPTO web site (http://seqdata.uspto.gov/?pageRequest=docDetail&DocID=US20110294680A1). An electronic copy of the table will also be available from the USPTO upon request and payment of the fee set forth in 37 CFR 1.19(b)(3).

Controls for occult stratification among the tested subjects and poor technical quality in the nominally-positive SNPs identified failed to provide alternative explanations for the positive results of comparisons between smokers and controls. Only 837 of the nominally-positive SNPs from the smoker-control comparisons displayed large allele frequency differences between European- and African-American control individuals. This number was smaller than the 2,223 SNPs that would be expected to have such properties if they were selected by chance. Only 158 of the nominally-positive SNPs from the smoker-control comparisons in these data lay among the SNPs that displayed the largest variation between pools in data from this and other studies using the same arrays. This number was also smaller than chance values. These comparisons thus fail to support the alternative hypotheses that either occult ethnic stratification in these samples or technical problems with assays for these SNPs provided the basis for the overall results.

In comparing data from successful versus unsuccessful quitters, 4,570 SNPs were identified whose allele frequencies differ between these two groups with t values for these differences that yield nominal p values<0.01. The nominally-positive SNPs from comparisons between successful versus unsuccessful quitters clustered together to extents much greater than expected by chance if their allelic frequencies were independent of each other (Monte Carlo p<0.00001). 944 of the 4,570 nominally-positive SNPs lay in 224 clusters in which each positive SNP lay within 100 Kb of at least one other positive SNP. Such clustering would be anticipated if many of these reproducibly-positive SNPs identified haplotypes that were present in different frequencies in the samples of successful versus unsuccessful quitters, but not if they represented chance independent observations. Clusters were defined as chromosomal sites where: 1) three or more reproducibly-positive SNPs were positioned within 0.1 Mb of each other and 2) reproducibly-positive SNPs assessed by two different array types were represented, so that all positive data did not come from just NspI or StyI arrays. The nominally-positive SNPs from successful versus unsuccessful quitter comparisons that clustered together on small chromosomal regions also clustered together in regions that are annotated as genes to extents much greater than chance if they represented independent observations (Monte Carlo p<0.00001 for both).

Controls for occult stratification among the tested subjects and poor technical quality in the nominally-positive SNPs identified failed to provide alternative explanations for the positive results of comparisons between successful and unsuccessful quitters. The SNPs that displayed the largest allele frequency differences between European- and African-American controls and the SNPs that displayed the largest between-pool variances did not overlap with those that distinguished successful versus unsuccessful quitters at levels significantly larger than those anticipated by chance (131 versus 114 and 143 versus 114, respectively).

### Example 2

### Molecular Genetics of Successful Smoking Cessation

Convergent genome-wide association studies of European-American participants in smoking cessation clinical trials from three centers were undertaken to identify replicated quit success genes. Genotypes from the participants who successfully abstained from smoking in a clinical smoking cessation trial were compared to genotypes from the participants who were unsuccessful (i.e., relapsed) in abstaining from smoking. See, Uhl et al. (2008) Arch Gen Psychiatry. 65:683-693, incorporated herein by reference as if set forth in its entirety.

Experimental Subjects

European American smokers responded to newspaper, flyer and television advertisements and/or to physician referrals for help in quitting smoking (Lerman et al. (2006) Neuropsychopharmacology 31:231-242; Liu et al. (2006), supra; David et al. (2007) Nicotine Tob. Res. 9:821-833). Subjects were 18-65 years of age, provided informed consents, were not pregnant or lactating, had no DSM-IV Axis I psychiatric disorder, reported no current use of treatment medications (e.g., bupropion or nicotine-containing products other than cigarettes) and described no contraindications for use of these medications. Individuals who were dependent on other addictive substances, current users of psychotropic medications and those diagnosed with cancer in the past 6 months were excluded. Subjects were enrolled in one of four randomized clinical trials for smoking cessation.

Sample I: (a) Double-blind placebo controlled trial of bupropion 300 mg/day or matching placebo for 10 weeks, or (b) open label trial of nicotine nasal spray versus nicotine patch for 8 weeks (Lerman et al. (2006), supra). Smoking status was assessed by telephone interview 0, 8 and 24 weeks after the targeted quit date using validated timeline follow-back methods (Brown et al. (1998) Psych. Add. Behav. 12:101-112). Abstinence was also assessed by measuring cotinine <15 ng/ml (bupropion trial) or CO (NRT trial). One hundred twenty-six individuals with biochemically-confirmed abstinence for at least the 7 days prior to both a) the end of treatment (8 weeks) and b) 24 week assessments were contrasted with 140 unsuccessful quitters who were not abstinent at either time point. Sample I was 55% female, averaged 45 years of age, reported smoking an average of 21 cigarettes/day and displayed FTND scores that averaged 5.4 prior to treatment. Eighty-nine percent described at least one prior effort to quit smoking.

Sample II: Clinical effectiveness study of NRT. Participants received either active 21 mg/day or placebo nicotine skin patches for two weeks before the targeted quit date. Participants also received mecamylamine 10 mg/day per os (po), prior to the target quit-smoking date in order to attenuate reinforcing effects of cigarette smoking. After the quit-date, participants were randomly assigned to groups that received mecamylamine 10 mg/day versus matching placebo and 21 mg/24 hours versus 42 mg/24 hours nicotine skin patch doses. Fifty-five individuals reported continuous abstinence from smoking when assessed 6 weeks after the quit date with CO confirmation, 79 were not abstinent. Sample II was 48% female, averaged 44 years of age, reported smoking an average of 30 cigarettes/day with FTND scores averaging 6.4 prior to treatment. Most of these individuals reported at least one prior quit attempt; there was an average of 4.4 quit attempts (Liu et al. (2006), supra).

Sample III: Double-blind placebo controlled trial of bupropion 300 mg or matching placebo for 10 weeks. Participants received 10 weeks of either placebo or bupropion (150 mg/day for the first 3 days, then 300 mg/day) with a target quit date one week following initiation of drug or placebo (David et al. (2007), supra). Smoking cessation was assessed using point abstinence, defined by self reports and saliva cotinine levels ≦15 ng/ml. Sixty individuals with biochemically-confirmed abstinence for at least the 7 days prior to both the end of treatment and 24 week assessments were contrasted with 90 unsuccessful quitters who were not abstinent at either time point. Sample III was 51% female, averaged 45 years of age, reported smoking an average of 25 cigarettes/day with FTND scores of 7.5 prior to treatment. Most of these individuals reported at least one prior quit attempt; there was an average of 5 quit attempts.

DNA Preparation, Pooling and Analysis

Genomic DNA was prepared from blood, carefully quantitated, combined into pools representing 13-20 quitter or nonquitter subjects, and analyzed as described by Uhl et al. (Am. J. Hum. Genet. 69:1290-1300, 2001). Allele frequencies for each SNP in each DNA pool were assessed based on hybridization to the “perfect match” cells from replicate experiments, as described by Liu et al. (2006), supra; and Johnson et al. (2006), supra). A “t” statistic for the differences between quitters and nonquitters was then generated (Liu et al. (2005) Proc. Natl. Acad. Sci. USA 102:11864-11869; Liu et al. (2006), supra; and Johnson et al. (2006), supra. “Nominally positive” SNPs displayed t values with p<0.01. Analyses focused on nominally-positive SNPs that clustered in small chromosomal regions in at least two samples, since this pattern of results would not be anticipated by chance but would be anticipated if haplotype blocks that contained multiple SNPs were present at differing frequencies in successful versus unsuccessful quitters.

Monte Carlo p values for the clustering of nominally-positive SNPs from one sample that lie within annotated genes and the convergence between these clusters and the data from at least one other sample were calculated based on 100,000 or 10,000 simulation trials. Each trial sampled a random set of SNPs from the database that contains the results from these studies and applied the same procedure that had been followed for the actual data analysis. The number of trials for which the results from the randomly-selected set of SNPs matched or exceeded the results actually observed from the SNPs identified in the instant study was tabulated. Empirical p values were calculated by dividing the number of trials for which the observed results were matched or exceeded by the total number of Monte Carlo simulation trials performed. This method examines the properties of the SNPs in the current dataset and thus should be relatively robust despite the uneven distribution of Affymetrix SNP markers across the genome, the slightly different complement of SNPs represented on the early access and commercial versions of the arrays, and the differing criteria for clustering applied to the larger Sample I and smaller Samples II and III.

Statistical power was estimated using the observed standard deviations and mean abuser/control differences from each sample, the program PS v2.1.31 (Dupont & Plummer (1990), supra) and α=0.05.

Controls for the possibility that quitter/non-quitter differences were due to occult ethnic differences or noisy assays compared the clustered SNPs that displayed nominally-positive results in the instant study to SNPs that displayed a) the largest differences among previously-studied African-American versus European-American control individuals, b) the largest differences among subsets of self-reported Caucasian control individuals recruited from different sites within the United Kingdom or c) the largest variation between pools in data from this and other studies that used the same arrays (see, Liu et al. (2006), supra).

Secondary analyses pooled data from placebo, bupropion and nicotine replacement-treated individuals from all three samples. For each SNP, t values for allele frequency differences between quitters versus nonquitters in bupropion versus placebo and NRT versus placebo comparisons were plotted. While there is no single statistical approach to such data, SNPs were sought that displayed nominally to highly significant impact on responses to at least one of these treatments, and were divided into those SNPs that displayed bupropion-selective, nicotine-replacement selective or nonselective effects. Single nucleotide polymorphisms that displayed t values corresponding to p<0.005 for NRT (t>3.69), bupropion (t>3.58) or both were identified.

The differences between the t values for NRT versus placebo and the t values for the differences between bupropion versus placebo for each SNP were calculated. The ⅓ of SNPs for which t values for NRT provided the largest positive differences from those for bupropion were defined as “NRT specific,” while the ⅓ of SNPs for which t values for nicotine replacement were similar to those for bupropion were defined as “nonspecific” and the ⅓ of SNPs for which t values for bupropion provided the largest positive differences from those for NRT were defined as “bupropion specific.” The genes that were identified by at least two SNPs were then tallied.

Results

There was modest variability in the allele frequency assessments that were based on hybridization to the “perfect match” cells on each of four arrays for DNA from each pool (Liu et al (2006), supra; Johnson et al. (2006), supra). Standard errors (SEMs) for the variation among the four replicate studies of each DNA pool were +/−0.037 for sample I, +/−0.035 for sample II and +/−0.048 for sample III. Standard errors for the variation among the pools studied for each phenotype group were +/−0.029 for sample I, +/−0.028 for sample II and +/−0.34 for sample III. These results support modest variation and are consistent with validation studies that reveal 0.95 correlations between pooled and individually-determined genotype frequencies using these arrays (Uhl et al. (2001), supra; Liu et al. (2005), supra; Liu et al. (2006), supra; Bang-Ce et al. (2004) Anal. Biochem. 333:72-78; Sham et al. (2002) Nat. Rev. Genet. 3:862-871; and Hinds et al. (2004) Hum. Genomics 1:421-434). Based on these variances, the power to detect 5% and 10% allele frequency differences was 0.71/0.45/0.46 and 0.99/0.95/0.96 in samples I, II and III, respectively.

In comparing data from successful versus unsuccessful quitters from samples I, II and III, 5,411, 4,539 and 4,894 SNPs were identified whose allele frequencies differed between these two groups with nominal p values<0.01. The nominally-positive SNPs from comparisons between successful versus unsuccessful quitters in each of these samples cluster together to extents much greater than expected by chance if their allelic frequencies were independent of each other (Monte Carlo p<0.00001). For Sample I, 1,434 of these 5,411 nominally-positive SNPs, lay in 308, 820 and 861 clusters in which each positive SNP lay within 0.1 Mb of at least two other positive SNPs with representation from SNPs on both array types. For Samples II and III, 2,258 of the 4,539 nominally-positive SNPs and 2,184 of the 4,894 nominally-positive SNPs lay in 820 and 861 clusters in which each positive SNP lay within 0.1 Mb of at least one other positive SNP.

Controls for occult stratification among the tested subjects and poor technical quality in the nominally-positive SNPs identified failed to provide alternative explanations for the positive results of comparisons between successful versus unsuccessful quitters. Neither SNPs that display the largest allele frequency differences between European- and African-American controls in previous assessments, SNPs that display the largest allele frequency differences between self-reported Caucasian United Kingdom samples, nor SNPs that display the largest between-pool variances in the current dataset overlap with those that distinguish successful versus unsuccessful quitters at levels larger than those anticipated by chance (40 versus 135, 0 versus 0 and 103 versus 105 for Sample I; 39 versus 114, 0 versus 0 and 115 versus 120 for Sample II and 35 versus 122, 0 versus 0 and 112 versus 95 for Sample III).

Table 1 includes genes where three or more (Sample I) or two or more (Samples II and III) nominally-positive SNPs cluster and clustered nominally-positive SNPs from at least one other sample are also present. Nominally-positive clustered SNPs from successful versus unsuccessful quitter comparisons from Samples I-III thus cluster together on small chromosomal regions to extents much greater than chance. The Monte Carlo p values for the replication/convergence for samples I and II, I and III and II and III are 0.00054, 0.0016 and 0.00063, respectively. Table 1 thus includes SNPs that display t values with p<0.01 in comparing successful versus unsuccessful quitters; cluster, so that at least three such SNPs on at least two array types (Sample I) or at least two such SNPs (Samples II and III) lie within 0.1 Mb of each other; identify annotated genes; and identify genes that contain clustered SNPs with p<0.01 in at least one other sample.

Eight, 23, 29, and 61 genes were identified by clustered positive SNPs that came from Samples I, II and III, I and II, I and III, and II and III, respectively. Monte Carlo p=0.0003 for this overall convergence. Twenty-one of the genes relate to cell adhesion, 24 are enzymes, 14 regulate transcription. Six encode receptors, 6 encode channels, 3 encode transporters and 4 encode receptor ligands. Two genes are involved with RNA processing, 2 with protein processing, 5 with intracellular signaling, 9 with cell structure, and 9 with unknown functions. Chromosomal regions not currently annotated as containing genes were found that contain at least two clustered nominally positive SNPs (within 0.1 Mb of each other) from at least two of the three samples described (Table 1).

In a secondary analysis, data from successful versus unsuccessful quitters from the groups treated with bupropion, NRT or placebo from all three centers were combined. The distribution oft values for the differences between bupropion versus placebo and the differences between NRT versus placebo indicate that some SNPs identify gene variants that do not influence responses to each of these treatments in the same way. Single nucleotide polymorphisms that displayed t values corresponding to p<0.005 for nicotine replacement, bupropion or both were identified and the differences between the t values for NRT versus placebo and the t values for the differences between bupropion versus placebo for each SNP were calculated. The ⅓ of SNPs for which t values for NRT provided the largest positive differences from those for bupropion were considered “NRT specific,” the 1/3 of SNPs for which t values for NRT were similar to those for bupropion were considered “nonspecific” and the ⅓ of SNPs for which t values for bupropion provided the largest positive differences from those for nicotine replacement were considered “bupropion specific.” Genes that were identified by at least two SNPs in these groups are included in Table 1. SNPs in the 41 “NRT specific,” 66 “nonspecific” and 26 “bupropion specific” genes had mean t values of 4.28 versus 1.71, 3.58 versus 2.67 and 1.71 versus 4.28 for NRT versus bupropion, respectively. Bupropion- and NRT-selective SNPs each clustered in small chromosomal regions with Monte Carlo p<0.00001.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A method for predicting success in addictive substance cessation in a subject comprising, detecting a single nucleotide polymorphism (SNP) in at least twenty nucleotide sequences set forth in SEQ ID NOs:1-14724 in a nucleic acid complement of said subject, wherein the presence of said SNP is correlated with an increased rate of success in addictive substance cessation.

2. The method of claim 1, wherein said addictive substance is selected from the group consisting of nicotine, alcohol, marijuana, cocaine, heroin, methamphetamine, ketamine, Ecstasy, oxycodone, codeine, morphine and combinations thereof.

3. The method of claim 1, wherein said addictive substance is nicotine.

4. The method of claim 1, wherein said subject presently is dependent on an addictive substance.

5. The method of claim 4, wherein said subject presently is dependent on nicotine.

6. The method of claim 1, in which detection of said SNP is carried out by a process selected from the group consisting of allele-specific probe hybridization, allele-specific primer extension, allele-specific amplification, sequencing, 5′ nuclease digestion, molecular beacon assay, oligonucleotide ligation assay, size analysis, single-stranded conformation polymorphism and combinations thereof.

7. A method for predicting success in nicotine cessation in a subject using a nicotine replacement source comprising, detecting a single nucleotide polymorphism (SNP) in at least twenty nucleotide sequences set forth in SEQ ID NOs:1-14724 in a nucleic acid complement of said subject, wherein the presence of said SNP is correlated with an increased rate of success in nicotine cessation in said subject using said nicotine replacement source.

8. The method of claim 7, wherein said nicotine replacement source is selected from the group consisting of a nicotine patch, a nicotine gum, a nicotine inhaler, or a nasal spray.

9. (canceled)

10. (canceled)

11. A method for predicting success in nicotine cessation in a subject using an antidepressant comprising, detecting a single nucleotide polymorphism (SNP) in one at least twenty nucleotide sequences according to claim 1 in a nucleic acid complement of said subject, wherein the presence of said SNP is correlated with an increased rate of success in nicotine cessation in said subject using said antidepressant.

12. The method of claim 11, wherein said antidepressant is bupropion.

13. A method for identifying a subject who has an increased risk of becoming dependent on an addictive substance, comprising detecting a single nucleotide polymorphism (SNP) in at least twenty nucleotide sequences according to claim 1 in a nucleic acid complement of said subject, wherein the presence of said SNP is correlated with an increased risk of becoming dependent on said addictive substance.

14. The method of claim 13, wherein said addictive substance is selected from the group consisting of nicotine, alcohol, marijuana, cocaine, heroin, methamphetamine, ketamine, Ecstasy, oxycodone, codeine, morphine and combinations thereof.

15. (canceled)

16. The method of claim 13, wherein said subject presently is dependent on an addictive substance.

17. The method of claim 13, in which detection of said SNP is carried out by a process selected from the group consisting of allele-specific probe hybridization, allele-specific primer extension, allele-specific amplification, sequencing, 5′ nuclease digestion, molecular beacon assay, oligonucleotide ligation assay, size analysis, single-stranded conformation polymorphism and combinations thereof.

18. A method for developing an individualized treatment regimen for addictive substance cessation in a subject dependent on an addictive substance comprising, detecting a single nucleotide polymorphisms (SNPs) in at least twenty nucleotide sequences according to claim 1 in a nucleic acid complement of said subject, wherein the presence of said one or more SNPs is correlated with an individualized treatment regimen.

19. The method of claim 18, wherein said addictive substance is selected from the group consisting of nicotine, alcohol, marijuana, cocaine, heroin, methamphetamine, ketamine, Ecstasy, oxycodone, codeine, morphine and combinations thereof.

20. (canceled)

21. The method of claim 18, wherein said subject presently is dependent on an addictive substance.

22. The method of claim 18, in which detection of said SNPs is carried out by a process selected from the group consisting of allele-specific probe hybridization, allele-specific primer extension, allele-specific amplification, sequencing, 5′ nuclease digestion, molecular beacon assay, oligonucleotide ligation assay, size analysis, single-stranded conformation polymorphism and combinations thereof.

23. An isolated nucleic acid molecule comprising at least 25 contiguous nucleotides selected from any one nucleotide sequence set forth in SEQ ID NOs:1 to 14724, or a complement thereof, wherein one of the nucleotides is a single nucleotide polymorphism (SNP).

24. (canceled)

25. A kit for detecting a single nucleotide polymorphism (SNP) in a nucleic acid, comprising the isolated nucleic acid molecule of claim 23.
